(19) United States
(12) Patent Application Publication
(10) Pub. No.: US 2026/0170322 A1
(43) Pub. Date: Jun. 18, 2026

(54) TRAINING MULTI-MODAL MODELS WITH BATCHES OF LIMITED MODALITY COMBINATIONS

(71) Applicant: NEC Corporation, Tokyo (JP)

(72) Inventors: Chenhui HUANG, Tokyo (JP); Kensuke WAGATA, Tokyo (JP); Fumiyuki NIHEY, Tokyo (JP); Yuki KOSAKA, Tokyo (JP); Pierre MACHART, Hamburg (DE); Giampaolo PILEGGI, Lainate (IT)

(21) Appl. No.: 18/983,355

(22) Filed: Dec. 17, 2024

(51) Int. Cl. G06N 3/08; G06N 3/0455; G16H 50/30

(52) U.S. Cl. CPC G06N 3/08; G06N 3/0455; G16H 50/30

(57) ABSTRACT

Training multi-modal models with batches of limited modality combinations is implemented by grouping training data samples into batches, each training data sample including data values corresponding to each data type, grouping data types into modalities of data types, generating modality lists, each modality list including one or more modalities, and performing, for each modality list to produce a multi-modal model for estimating a result from an incomplete data sample, applying a probabilistic encoder to the data values corresponding to the modality in a batch to obtain a feature encoding, integrating the feature encoding of each modality to produce latent variables, applying a logistic regression decoder to the latent variables to estimate the result, determining a cost and a divergence, and adjusting parameters of the probabilistic encoders and the logistic regression decoder based on the cost and the divergence.

### FIELD

The present disclosure relates to training multi-modal models with batches of limited modality combinations.

### BACKGROUND

Predicting the risk of lifestyle-related diseases can contribute to the prevention of diseases. Diverse (multi-modal) body measurement data has recently become available in large-scale quantities. Individuals who become aware of a significant risk of a certain lifestyle-related disease based on general body measurement data may have better opportunities to prevent the disease.

### SUMMARY

Training multi-modal models with batches of limited modality combinations is implemented by grouping training data samples among a plurality of training data samples into a plurality of batches of training data samples, each training data sample including data values corresponding to each of a plurality of data types, grouping data types among the plurality of data types into a plurality of modalities of data types, generating a plurality of modality lists, each modality list including one or more modalities, performing, for each modality list among the plurality of modality lists to produce a multi-modal model for estimating a result from an incomplete data sample, applying, for each modality in the modality list, a probabilistic encoder to the data values corresponding to the modality in a batch of training samples among the plurality of batches to obtain a feature encoding corresponding to the modality, integrating the feature encoding of each modality in the modality list to produce latent variables, applying a logistic regression decoder to the latent variables to estimate the result, determining a cost by comparing the estimated result with a ground truth, determining a divergence by comparing the latent variables with a multi-dimensional Gaussian distribution, and adjusting parameters of the probabilistic encoders and the logistic regression decoder based on the cost and the divergence.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a schematic diagram of a multi-modal model, according to at least some embodiments of the subject disclosure.

FIG. 2 is a schematic diagram of training data sample of general body measurements, according to at least some embodiments of the subject disclosure.

FIG. 3 is an operational flow for training multi-modal models with batches of limited modality combinations, according to at least some embodiments of the subject disclosure.

FIG. 4 is schematic diagram of modalities of data types, according to at least some embodiments of the subject disclosure.

FIG. 5 is schematic diagram of modality combinations, according to at least some embodiments of the subject disclosure.

FIG. 6 is schematic diagram of modality lists, according to at least some embodiments of the subject disclosure.

FIG. 7 is an operational flow for producing a multi-modal model, according to at least some embodiments of the subject disclosure.

FIG. 8 is an operational flow for applying model to training data samples, according to at least some embodiments of the subject disclosure.

FIG. 9 is an operational flow for adjusting model parameters, according to at least some embodiments of the subject disclosure.

FIG. 10 is a block diagram of a hardware configuration for training multi-modal models with batches of limited modality combinations, according to at least some embodiments of the subject disclosure.

### DETAILED DESCRIPTION

The following disclosure provides many different embodiments, or examples, for implementing different features of the provided subject matter. Specific examples of components, values, operations, materials, arrangements, or the like, are described below to simplify the present disclosure. These are, of course, merely examples and are not intended to be limiting. Other components, values, operations, materials, arrangements, or the like, are contemplated. In addition, the present disclosure may repeat reference numerals and/or letters in the various examples. This repetition is for the purpose of simplicity and clarity and does not in itself dictate a relationship between the various embodiments and/or configurations discussed.

In applications known to the inventors, it is unlikely that all modalities will always be available. One technique known to the inventors for training a multi-modal model to be effective even in the presence of missing modalities is to incorporate all possible combinations of modalities into the loss function during training. However, as the number of modalities increases, the number of combinations increases exponentially according to the following formula:

$$n = {2}^{k} - 1 \qquad EQ . 1$$

where n is the number of combinations, and k is the number of modalities, leading to increased computational resource requirements.

In at least some embodiments of the subject disclosure, only a certain number of combinations are incorporated into the loss function during batch training of a multi-modal model.

By training multi-modal models with limited modality combinations in accordance with at least some embodiments of the subject disclosure, the computational resource requirement is reduced yet the accuracy of the trained multi-modal model is nearly the same as multi-modal models trained with all possible combinations of modalities.

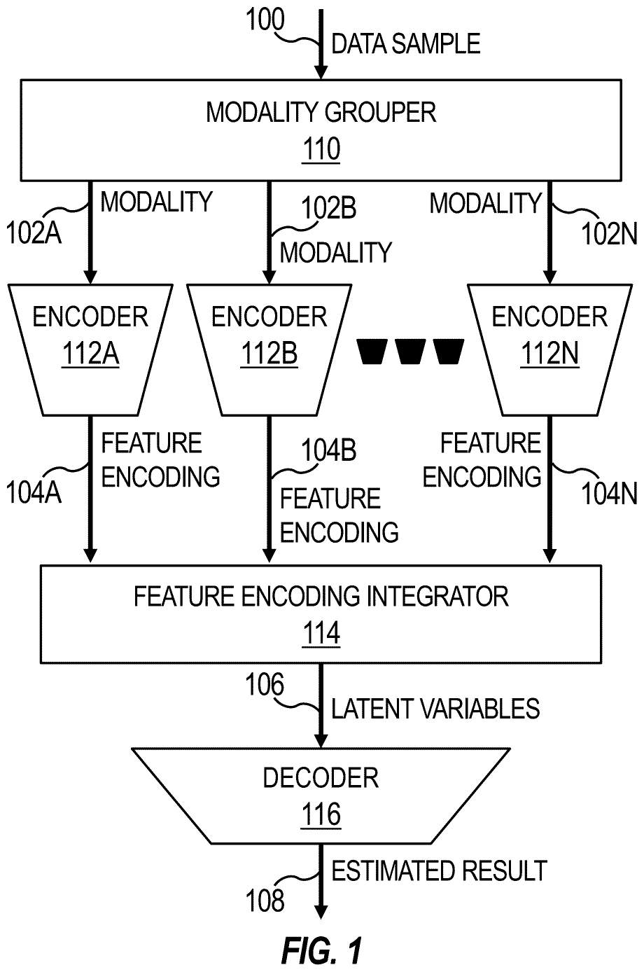

FIG. 1 is a schematic diagram of a multi-modal model, according to at least some embodiments of the subject disclosure. The multi-modal model includes modality grouper 110, encoder 112A, 112B, and 112N, feature encoding integrator 114, decoder 116, data sample 100, modality 102A, 102B, and 102N, feature encoding 104A, 104B, and 104N, latent variables 106, and estimated result 108.

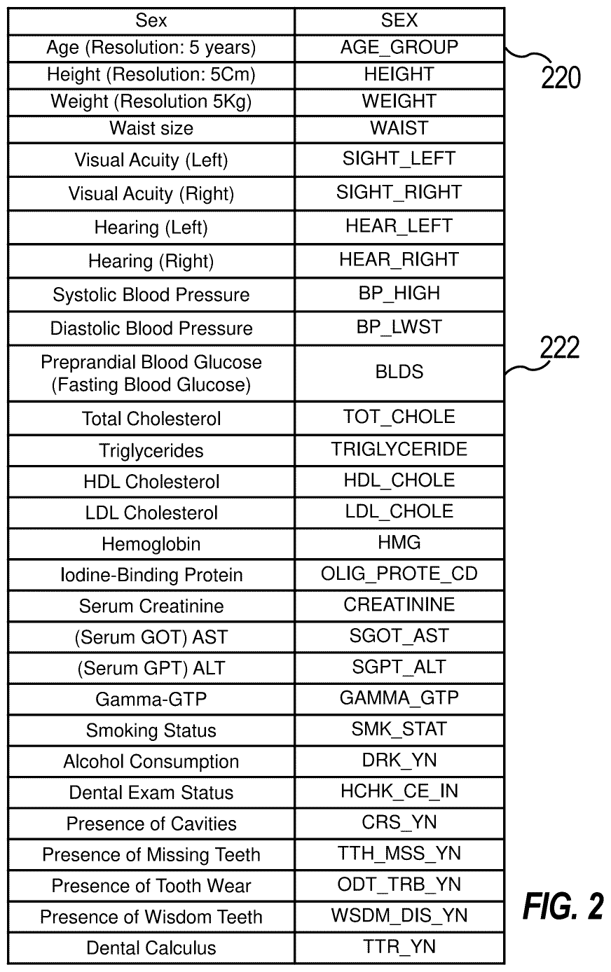

Data sample 100 is an input to the multi-modal model. In at least some embodiments, data sample 100 represents individual data points that contain various body measurements and their values. In at least some embodiments, data sample 100 is as described with respect to FIG. 2.

Modality grouper 110 is a component of the multi-modal model. In at least some embodiments, modality grouper 110 is of the type implemented in data management systems and preprocessing tools that handle diverse datasets. In at least some embodiments, modality grouper 110 is configured for organizing health data and preparing datasets for analysis. In at least some embodiments, modality grouper 110 is configured for categorizing data types into distinct modalities. In at least some embodiments, modality grouper 110 is configured to group data types into modalities.

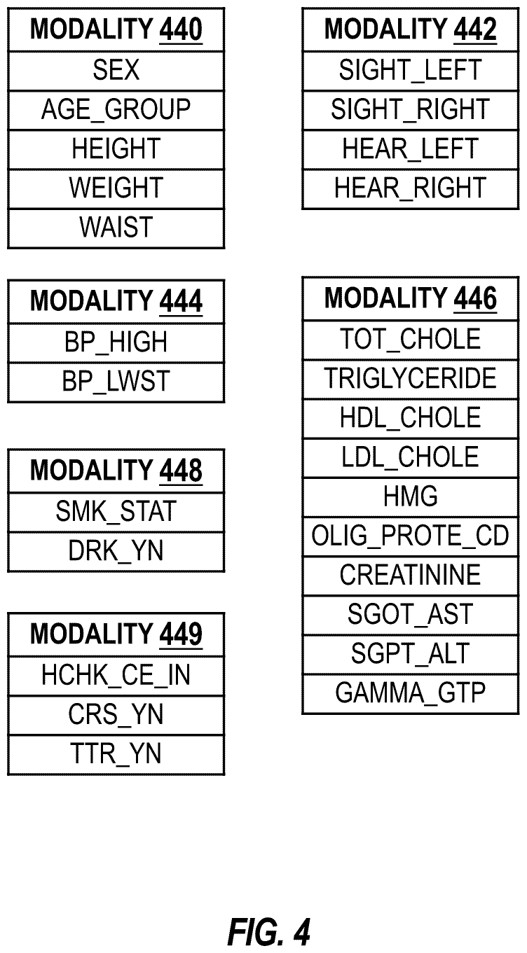

Modality 102A, 102B, and 102N are output of modality grouper 110 and input to encoders 112A, 112B, and 112N. In at least some embodiments, modality 102A, 102B, and 102N are groupings of data values from data sample 100. In at least some embodiments, modality 102A, 102B, and 102N are as described with respect to FIG. 4.

Encoders 112A, 112B, and 112N are components of the multi-modal model. In at least some embodiments, encoders 112A, 112B, and 112N are of the type typically utilized in machine learning and are designed for feature extraction. In at least some embodiments, encoders 112A, 112B, and 112N are configured for transforming raw data into usable features. In at least some embodiments, encoders 112A, 112B, and 112N are configured for encoding data values into feature representations that capture essential characteristics of the input data. In at least some embodiments, encoders 112A, 112B, and 112N are configured for handling different data types, such as numerical and categorical data, and performing dimensionality reduction. In at least some embodiments, encoders 112A, 112B, and 112N are trained to provide an integrator, such as feature encoding integrator 114, with feature encodings that represent the respective modality.

Feature encodings 104A, 104B, and 104N are output of encoders 112A, 112B, and 112N and input to feature encoding integrator 114. In at least some embodiments, feature encodings 104A, 104B, and 104N include essential characteristics of the input data. In at least some embodiments, feature encodings 104A, 104B, and 104N represent an average and a variance.

Feature encoding integrator 114 is a component of the multi-modal model. In at least some embodiments, feature encoding integrator 114 is of the type implemented in data fusion frameworks and statistical analysis tools that combine multiple data sources. In at least some embodiments, feature encoding integrator 114 is configured for combining feature encodings from various modalities to produce latent variables. In at least some embodiments, feature encoding integrator 114 is configured to receive feature encodings, such as feature encodings 104A, 104B, and 104N, and to transmit resulting latent variables, such as latent variables 106.

Latent variables 106 is output of feature encoding integrator 114 and input to decoder 116. In at least some embodiments, latent variables 106 are of the type represented in latent variable models and statistical modeling tools that analyze complex data relationships. In at least some embodiments, latent variables 106 represent underlying patterns in data to facilitate predictive modeling.

Decoder 116 is a component of the multi-modal model. In at least some embodiments, decoder 116 is of the type utilized in predictive modeling software and risk assessment tools that generate outcomes based on input data. In at least some embodiments, decoder 116 is configured for estimating disease risk and interpreting model predictions. In at least some embodiments, decoder 116 is configured for decoding latent variables to produce estimated outcomes, often using logistic regression techniques. In at least some embodiments, decoder 116 is configured to receive latent variables, such as latent variables 106, and transmit estimated results, such as estimated result 108.

Estimated result 108 is output of the multi-modal model. In at least some embodiments, estimated result 108 represents a likelihood of a result. In at least some embodiments, estimated result 108 represents an individual disease risk. In at least some embodiments, once the multi-modal model is trained, an operator can apply the multi-modal model to a live data sample including data values corresponding to less than all of the plurality of data types.

FIG. 2 is a schematic diagram of training data sample of general body measurements, according to at least some embodiments of the subject disclosure. The training data sample of general body measurements includes data values, such as data value 220 and target data value 222. Each data value represents a data type. For example, data value 220 has a value of AGE_GROUP and represents a data type of “Age (Resolution: 5 years)”. In at least some embodiments, all of the data values are used as input to train a multi-modal model to predict a lifestyle-related disease, except for the one or more data values that directly indicate the lifestyle-related disease. Target data value 222 represents the data type of “Preprandial Blood Glucose (Fasting Blood Glucose)”, which is directly indicative of diabetes. In at least some embodiments, to train a multi-modal model to estimate the risk of diabetes, all of the data values of the training data sample are used as input except for target data value 222. In at least some embodiments, to train a multi-modal model to estimate the risk of diabetes, target data value 222 is used as the ground truth in order to determine the loss.

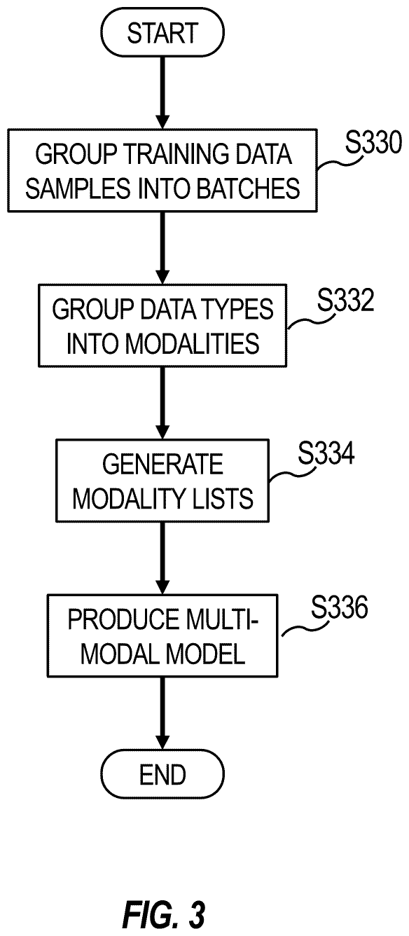

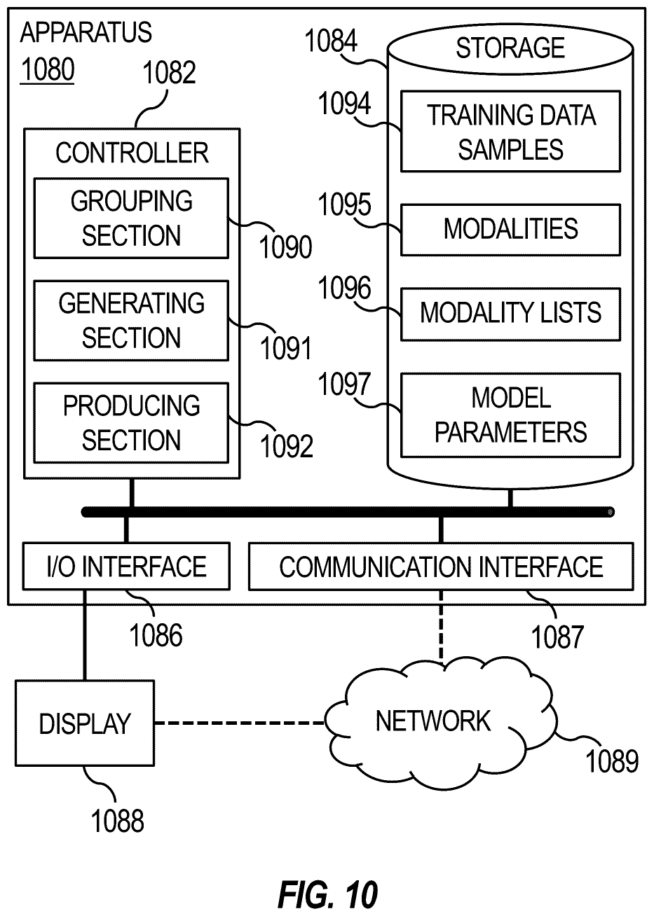

FIG. 3 is an operational flow for training multi-modal models with batches of limited modality combinations, according to at least some embodiments of the subject disclosure. In at least some embodiments, the operational flow provides a method of training multi-modal models with batches of limited modality combinations. In at least some embodiments, the method is performed by a controller of an apparatus, such as controller 1082 of apparatus 1080 of FIG. 10, described hereinafter.

At S330, the controller or a section thereof groups the training data samples into batches. In at least some embodiments, the controller shuffles training data samples, defines batch size, and assigns samples to batches. In at least some embodiments, the controller utilizes a batch size parameter to organize the data effectively. In at least some embodiments, the output produced consists of batches of training samples ready for processing in subsequent operations. In at least some embodiments, the controller performs grouping according to variable characteristics of batch size and method of shuffling. In at least some embodiments, varying these characteristics leads to faster training times with larger batch sizes, but also affects the model's ability to generalize in response to batches that are not representative of the overall dataset. In at least some embodiments, the controller groups training data samples among a plurality of training data samples into a plurality of batches of training data samples, each training data sample including data values corresponding to each of a plurality of data types. In at least some embodiments, each training data sample among the plurality of training data samples corresponds to a person, and wherein each data type among the plurality of data types is a body measurement.

At S332, the controller or a section thereof groups the data types into modalities. In at least some embodiments, the controller identifies data types. In at least some embodiments, the controller categorizes the data types into modalities. In at least some embodiments, the controller creates a mapping of these modalities. In at least some embodiments, the controller relies on a list of data types and predetermined modality definitions. In at least some embodiments, the controller groups data types among the plurality of data types into a plurality of modalities of data types.

At S334, the controller or a section thereof generates the modality lists. In at least some embodiments, the controller determines combinations of modalities. In at least some embodiments, the generating the plurality of modality lists includes determining possible combinations of modalities. In at least some embodiments, the controller creates each list based on a predetermined number of the combinations. In at least some embodiments, listed modalities of each modality list among the plurality of modality lists are included in a predetermined number of combinations of modalities. In at least some embodiments, the controller stores the modality lists in memory. In at least some embodiments, the generating the plurality of modality lists includes storing the plurality of modality lists in a memory. In at least some embodiments, the controller selects combinations for each list to evenly distribute modalities among the lists. In at least some embodiments, the controller generates a plurality of modality lists, each modality list including one or more modalities. In at least some embodiments, the plurality of modality lists include even distributions of modalities among the plurality of modalities.

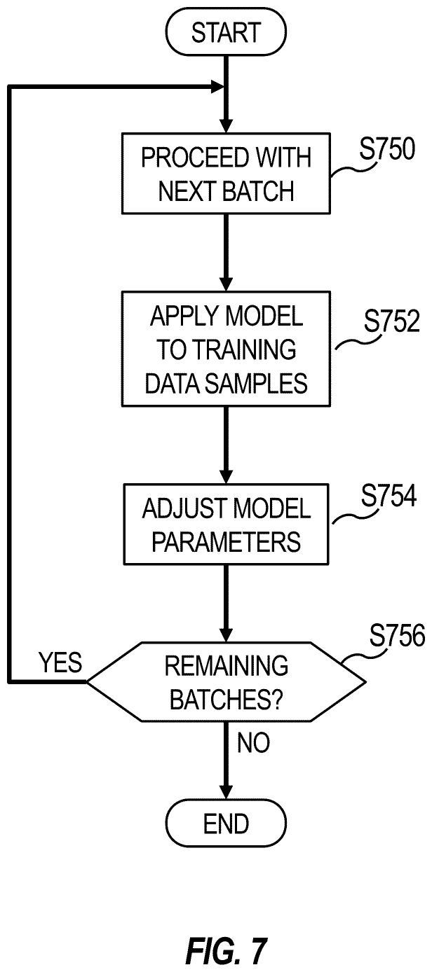

At S336, the controller or a section thereof produces the multi-modal model. In at least some embodiments, the controller applies the multi-modal model to the training data samples, calculates loss based on a comparison of the estimated result output from the multi-modal model with the ground truth of the training data samples, and adjusts parameters of the multi-modal model according to the calculated loss. In at least some embodiments, the controller performs operations for each modality list among the plurality of modality lists to produce a multi-modal model for estimating a result from an incomplete data sample. In at least some embodiments, the controller performs the operational flow of FIG. 7.

FIG. 4 is schematic diagram of modalities of data types, according to at least some embodiments of the subject disclosure. The modalities of data types include modalities 440, 442, 444, 446, 448, and 449. In at least some embodiments, modalities 440, 442, 444, 446, 448, and 449 include all of the data types of the training data samples except for the one or more target data types.

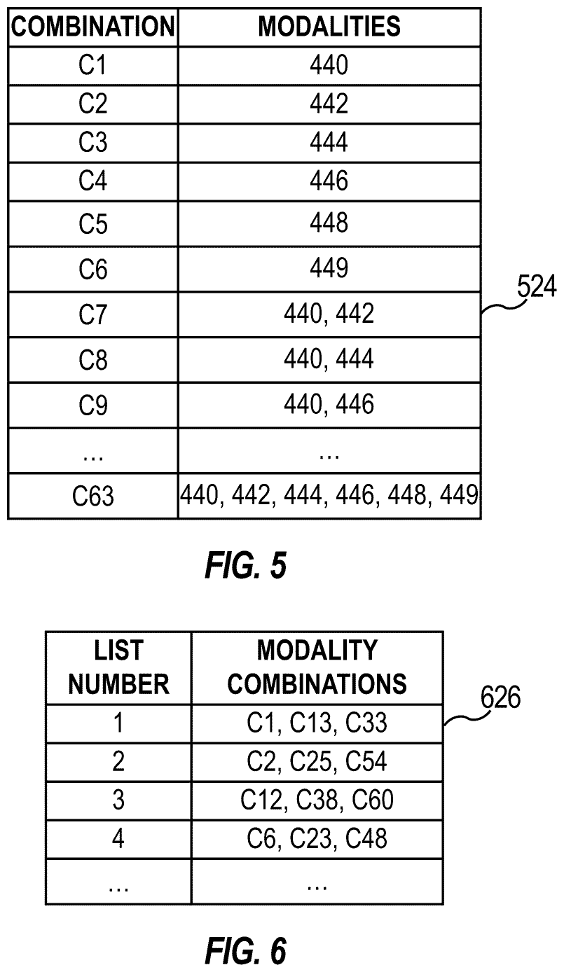

FIG. 5 is schematic diagram of modality combinations, according to at least some embodiments of the subject disclosure. The modality combinations include all possible combinations of modalities, such as modality combination 524. Modality combination 524 includes modality 440 and modality 442. In at least some embodiments, the number of modality combinations is calculated according to EQ. 1, described above. In at least some embodiments, a combination can include a single modality, two modalities, three modalities, etc., and including one combination with all modalities.

FIG. 6 is schematic diagram of modality lists, according to at least some embodiments of the subject disclosure. In at least some embodiments, each of the modality lists, such as modality list 626, include a list number and combinations. In at least some embodiments, each modality list includes a predetermined number of combinations. Modality list 626 includes three combinations, C1, C13, and C33. Combination C1 includes modality 440, combination C13 would include modality 442 and modality 446, and combination C33 would include modality 444, modality 448, and modality 449.

FIG. 7 is an operational flow for producing a multi-modal model, according to at least some embodiments of the subject disclosure. In at least some embodiments, the operational flow provides a method of producing a multi-modal model. In at least some embodiments, the method is performed by a controller of a apparatus, such as controller 1082 of apparatus 1080 of FIG. 10, described hereinafter.

At S750, the controller or a section thereof proceeds with the next batch. In at least some embodiments, the controller proceeds with the next batch of training data samples by checking for any remaining batches and loading the next set of training data samples. In at least some embodiments, the controller utilizes a batch data structure that organizes the training data samples for processing.

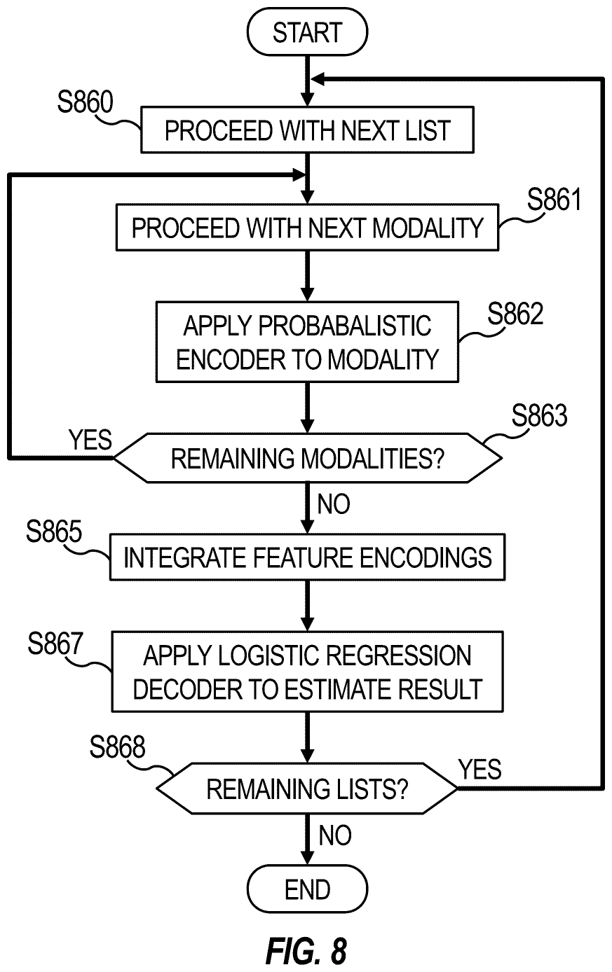

At S752, the controller or a section thereof applies the model to the training data samples. In at least some embodiments, the controller applies the multi-modal model to the training data samples by inputting the data values from the training data samples into the multi-modal model and executing a forward pass to output an estimated result. In at least some embodiments, the controller performs the operational flow of FIG. 8, described hereinafter.

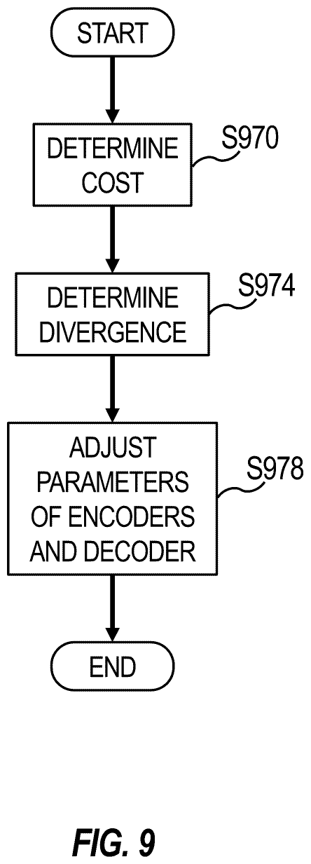

At S754, the controller or a section thereof adjusts the model parameters. In at least some embodiments, the controller adjusts the model parameters by computing gradients and updating the weights based on the loss function. In at least some embodiments, the controller utilizes the following loss function for one modality:

$${J}_{IB} = \frac{1}{N} {\sum }_{n = 1}^{N} {𝔼}_{∈ \sim p ( ∈ )} [ - log q ( {y}_{n} \vert f ( {x}_{n} , ∈ ) ) ] + β KL [ p ( Z \vert {x}_{n} ) , r ( Z ) ] \qquad EQ . 2$$

where J_{IB} is the loss, N is the number of training data samples in the batch, where ϵ˜N (0,I) is an auxiliary Gaussian noise variable, KL is the Kullback-Leibler divergence and f is a vector-valued parametric deterministic encoding function,), assuming q(y|z) and r(z) are variational approximations of the true p(y|z) and p(z), respectively. In at least some embodiments, the controller determines loss for each modality as

$${J}_{MVIB} ( {x}^{A} ) + {J}_{MVIB} ( {x}^{M} ) + {J}_{MVIB} ( {x}^{A} , {x}^{M} ) \qquad EQ . 3$$

where x^{A} and x^{B} are the modalities in the combinations of the modality list. In at least some embodiments, the controller determines loss based on cost J_{AI} and divergence J_{BI} as follows:

$${J}_{I} = a * {J}_{AI} + b * {J}_{BI} \qquad EQ . 4$$

where J_{I} is the total loss, and a and b are hyperparameter coefficients. In at least some embodiments, the controller performs the operational flow of FIG. 9, described hereinafter.

At S756, the controller or a section thereof determines whether there are remaining batches. In at least some embodiments, the controller checks for remaining batches by evaluating the batch count and determining whether to continue or end the training process. In at least some embodiments, the controller utilizes a batch counter. In response to the controller determining that there are remaining batches, the operational flow returns to proceed with the next batch at S750. In response to the controller determining that there are no remaining batches, the operational flow ends.

FIG. 8 is an operational flow for applying model to training data samples, according to at least some embodiments of the subject disclosure. In at least some embodiments, the operational flow provides a method of applying model to training data samples. In at least some embodiments, the method is performed by a controller of a apparatus, such as controller 1082 of apparatus 1080 of FIG. 10, described hereinafter.

At S860, the controller or a section thereof proceeds with the next list. In at least some embodiments, the controller proceeds by identifying the next modality list to be processed and loading the corresponding modalities.

At S861, the controller or a section thereof proceeds with the next modality. In at least some embodiments, the controller selects the next modality from the current modality list and prepares the associated data values from the training data samples for processing.

At S862, the controller or a section thereof applies a probabilistic encoder to the modality. In at least some embodiments, the controller applies a corresponding probabilistic encoder to the selected modality. In at least some embodiments, the controller determines which probabilistic encoder corresponds to the selected modality. In at least some embodiments, the controller applies, for each modality in the modality list, a probabilistic encoder to the data values corresponding to the modality in a batch of training samples among the plurality of batches to obtain a feature encoding corresponding to the modality.

At S863, the controller or a section thereof determines whether there are remaining modalities. In at least some embodiments, the controller checks for any remaining modalities to process. In at least some embodiments, this involves evaluating the list of modalities to determine if additional modalities are available for processing. In response to the controller determining that there are remaining modalities, the operational flow returns to proceed with the next modality at S861. In response to the controller determining that there are no remaining modalities, the operational flow proceeds to feature encodings integration at S865.

At S865, the controller or a section thereof integrates the feature encodings. In at least some embodiments, the controller integrates the feature encodings obtained from each modality in the modality list to generate latent variables. In at least some embodiments, the latent variables encapsulate the integrated information from the modalities. In at least some embodiments, the controller integrates the feature encoding of each modality in the modality list to produce latent variables.

At S876, the controller or a section thereof applies the logistic regression decoder to estimate the result. In at least some embodiments, the controller applies a logistic regression decoder to the latent variables to estimate the result. In at least some embodiments, the controller applies the logistic regression decoder to the latent variables to estimate a risk of a lifestyle-related disease. In at least some embodiments, the logistic regression decoder estimates a risk score for a lifestyle-related disease.

At S868, the controller or a section thereof determines whether there are remaining lists. In at least some embodiments, the controller checks for any remaining modality lists to process. In response to the controller determining that there are remaining lists, the operational flow returns to proceed with the next modality list at S860. In response to the controller determining that there are no remaining lists, the operational flow ends.

FIG. 9 is an operational flow for adjusting model parameters, according to at least some embodiments of the subject disclosure. In at least some embodiments, the operational flow provides a method of adjusting model parameters. In at least some embodiments, the method is performed by a controller of a apparatus, such as controller 1082 of apparatus 1080 of FIG. 10, described hereinafter.

At S970, the controller or a section thereof determines the cost. In at least some embodiments, the controller computes the cost Jar according to the loss function in EQ. 2 and EQ. 3. In at least some embodiments, the controller determines a cost by comparing the estimated result with a ground truth.

At S974, the controller or a section thereof determines the divergence. In at least some embodiments, the controller computes the cost JB according to the loss function in EQ. 2 and EQ. 3. In at least some embodiments, the controller determines a divergence by comparing the latent variables with a multi-dimensional Gaussian distribution.

At S978, the controller or a section thereof adjusts the parameters of the encoders and decoder. In at least some embodiments, the controller updates the parameters of both the encoder and decoder components of the model. In at least some embodiments, the controller applies an optimization algorithm to refine the parameters based on the computed cost and divergence. In at least some embodiments, the controller utilizes backpropagation and gradient descent to adjust the parameters. In at least some embodiments, the controller adjusts parameters of the probabilistic encoders and the logistic regression decoder based on the cost and the divergence.

FIG. 10 is a block diagram of a hardware configuration for training multi-modal models with batches of limited modality combinations, according to at least some embodiments of the subject disclosure. The hardware configuration includes apparatus 1080, which interacts with display 1088 directly or through network 1089. In at least some embodiments, display 1088 is a touch screen or any other device configured for input and output. In at least some embodiments, network 1089 is an ethernet network, or any other wired or wireless network or a combination thereof. In at least some embodiments, apparatus 1080 is a computer or other computing device that receives input or commands from display 1088. In at least some embodiments, apparatus 1080 is integrated with display 1088. In at least some embodiments, apparatus 1080 is a computer system that executes computer-readable instructions to perform operations for training multi-modal models with batches of limited modality combinations.

Apparatus 1080 includes controller 1082, storage 1084, input/output interface 1086, and communication interface 1087. In at least some embodiments, controller 1082 includes a processor or programmable circuitry executing instructions to cause the processor or programmable circuitry to perform operations according to the instructions. In at least some embodiments, controller 1082 includes analog or digital programmable circuitry, or any combination thereof. In at least some embodiments, controller 1082 includes physically separated storage or circuitry that interacts through communication. In at least some embodiments, storage 1084 includes a non-volatile computer-readable medium capable of storing executable and non-executable data for access by controller 1082 during execution of the instructions. In at least some embodiments, communication interface 1087 transmits and receives data from network 1089. In at least some embodiments, input/output interface 1086 connects to various input and output units, such as display 1088, via a parallel port, a serial port, a keyboard port, a mouse port, a monitor port, and the like to accept commands and present information. In some embodiments, storage 1084 is external from apparatus 1080.

Controller 1082 includes grouping section 1090, generating section 1091, and producing section 1092. Storage 1084 includes training data samples 1094, modalities 1095, modality lists 1096, and model parameters 1097.

Grouping section 1090 is the circuitry or instructions of controller 1082 configured to group training data samples into batches and group data types into modalities. In at least some embodiments, grouping section 1090 is configured to group training data samples among a plurality of training data samples into a plurality of batches of training data samples, each training data sample including data values corresponding to each of a plurality of data types. In at least some embodiments, grouping section 1090 is configured to group data types among the plurality of data types into a plurality of modalities of data types. In at least some embodiments, grouping section 1090 utilizes storage 1084 to read or record information, such as training data samples 1094 and modalities 1095. In at least some embodiments, grouping section 1090 includes sub-sections for performing additional functions, as described in the foregoing flow charts. In at least some embodiments, such sub-sections are referred to by a name associated with a corresponding function.

Generating section 1091 is the circuitry or instructions of controller 1082 configured to generate modality lists. In at least some embodiments, generating section 1091 is configured to generate a plurality of modality lists, each modality list including one or more modalities. In at least some embodiments, generating section 1091 utilizes storage 1084 to read or record information, such as modalities 1095 and modality lists 1096. In at least some embodiments, generating section 1091 includes sub-sections for performing additional functions, as described in the foregoing flow charts. In at least some embodiments, such sub-sections are referred to by a name associated with a corresponding function.

Producing section 1092 is the circuitry or instructions of controller 1082 configured to produce multi-modal models. In at least some embodiments, producing section 1092 is configured to produce a multi-modal model for estimating a result from an incomplete data sample. In at least some embodiments, producing section 1092 utilizes storage 1084 to read or record information, such as model parameters 1097. In at least some embodiments, producing section 1092 includes sub-sections for performing additional functions, as described in the foregoing flow charts. In at least some embodiments, such sub-sections are referred to by a name associated with a corresponding function.

In at least some embodiments, the apparatus is another device capable of processing logical functions in order to perform the operations herein. In at least some embodiments, the controller and the storage need not be entirely separate devices, but share circuitry or one or more computer-readable mediums. In at least some embodiments, the storage includes a hard drive storing both the computer-executable instructions and the data accessed by the controller, and the controller includes a combination of a central processing unit (CPU) and RAM, in which the computer-executable instructions are able to be copied in whole or in part for execution by the CPU during performance of the operations herein.

In at least some embodiments where the apparatus is a computer, a program that is installed in the computer is capable of causing the computer to function as or perform operations associated with apparatuses of the embodiments described herein. In at least some embodiments, such a program is executable by a processor to cause the computer to perform certain operations associated with some or all of the blocks of flowcharts and block diagrams described herein.

At least some embodiments are described with reference to flowcharts and block diagrams whose blocks represent (1) steps of processes in which operations are performed or (2) sections of hardware responsible for performing operations. In at least some embodiments, certain steps and sections are implemented by dedicated circuitry, programmable circuitry supplied with computer-readable instructions stored on computer-readable media, and/or processors supplied with computer-readable instructions stored on computer-readable media. In at least some embodiments, dedicated circuitry includes digital and/or analog hardware circuits and include integrated circuits (IC) and/or discrete circuits. In at least some embodiments, programmable circuitry includes reconfigurable hardware circuits comprising logical AND, OR, XOR, NAND, NOR, and other logical operations, flip-flops, registers, memory elements, etc., such as field-programmable gate arrays (FPGA), programmable logic arrays (PLA), etc.

In at least some embodiments, the computer-readable medium includes a tangible device that is able to retain and store instructions for use by an instruction execution device. In some embodiments, the computer-readable medium includes, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

While embodiments of the present invention have been described, the technical scope of any subject matter claimed is not limited to the above described embodiments. Persons skilled in the art would understand that various alterations and improvements to the above-described embodiments are possible. Persons skilled in the art would also understand from the scope of the claims that the embodiments added with such alterations or improvements are included in the technical scope of the invention.

The operations, procedures, steps, and stages of each process performed by an apparatus, system, program, and method shown in the claims, embodiments, or diagrams are able to be performed in any order as long as the order is not indicated by “prior to,” “before,” or the like and as long as the output from a previous process is not used in a later process. Even if the process flow is described using phrases such as “first” or “next” in the claims, embodiments, or diagrams, such a description does not necessarily mean that the processes must be performed in the described order.

Training multi-modal models with batches of limited modality combinations is implemented by grouping training data samples among a plurality of training data samples into a plurality of batches of training data samples, each training data sample including data values corresponding to each of a plurality of data types, grouping data types among the plurality of data types into a plurality of modalities of data types, generating a plurality of modality lists, each modality list including one or more modalities, performing, for each modality list among the plurality of modality lists to produce a multi-modal model for estimating a result from an incomplete data sample, applying, for each modality in the modality list, a probabilistic encoder to the data values corresponding to the modality in a batch of training samples among the plurality of batches to obtain a feature encoding corresponding to the modality, integrating the feature encoding of each modality in the modality list to produce latent variables, applying a logistic regression decoder to the latent variables to estimate the result, determining a cost by comparing the estimated result with a ground truth, determining a divergence by comparing the latent variables with a multi-dimensional Gaussian distribution, and adjusting parameters of the probabilistic encoders and the logistic regression decoder based on the cost and the divergence.

In at least some embodiments, training multi-modal models with batches of limited modality combinations is further implemented by applying the model to a live data sample including data values corresponding to less than all of the plurality of data types. In at least some embodiments, the plurality of modality lists include even distributions of modalities among the plurality of modalities. In at least some embodiments, the generating the plurality of modality lists includes determining possible combinations of modalities. In at least some embodiments, listed modalities of each modality list among the plurality of modality lists are included in a predetermined number of combinations of modalities. In at least some embodiments, the generating the plurality of modality lists includes storing the plurality of modality lists in a memory. In at least some embodiments, the feature encoding represents an average and a variance. In at least some embodiments, each training data sample among the plurality of training data samples corresponds to a person, and wherein each data type among the plurality of data types is a body measurement. In at least some embodiments, the applying the logistic regression decoder to the latent variables is to estimate a risk of a lifestyle-related disease.

Training multi-modal models with batches of limited modality combinations is implemented by grouping training data samples among a plurality of training data samples into a plurality of batches of training data samples, each training data sample including data values corresponding to each of a plurality of data types, grouping data types among the plurality of data types into a plurality of modalities of data types, generating a plurality of modality lists, each modality list including one or more modalities, performing, for each modality list among the plurality of modality lists to produce a multi-modal model for estimating a result from an incomplete data sample, applying, for each modality in the modality list, a probabilistic encoder to the data values corresponding to the modality in a batch of training samples among the plurality of batches to obtain a feature encoding corresponding to the modality, integrating the feature encoding of each modality in the modality list to produce latent variables, applying a logistic regression decoder to the latent variables to estimate the result, determining a cost by comparing the estimated result with a ground truth, determining a divergence by comparing the latent variables with a multi-dimensional Gaussian distribution, and adjusting parameters of the probabilistic encoders and the logistic regression decoder based on the cost and the divergence.

In at least some embodiments, training multi-modal models with batches of limited modality combinations further includes applying the model to a live data sample including data values corresponding to less than all of the plurality of data types. In at least some embodiments, the plurality of modality lists include even distributions of modalities among the plurality of modalities. In at least some embodiments, the generating the plurality of modality lists includes determining possible combinations of modalities. In at least some embodiments, listed modalities of each modality list among the plurality of modality lists are included in a predetermined number of combinations of modalities. In at least some embodiments, the generating the plurality of modality lists includes storing the plurality of modality lists in a memory.

Training multi-modal models with batches of limited modality combinations is implemented by a controller including circuitry configured to perform operations comprising, grouping training data samples among a plurality of training data samples into a plurality of batches of training data samples, each training data sample including data values corresponding to each of a plurality of data types, grouping data types among the plurality of data types into a plurality of modalities of data types, generating a plurality of modality lists, each modality list including one or more modalities, performing, for each modality list among the plurality of modality lists to produce a multi-modal model for estimating a result from an incomplete data sample, applying, for each modality in the modality list, a probabilistic encoder to the data values corresponding to the modality in a batch of training samples among the plurality of batches to obtain a feature encoding corresponding to the modality, integrating the feature encoding of each modality in the modality list to produce latent variables, applying a logistic regression decoder to the latent variables to estimate the result, determining a cost by comparing the estimated result with a ground truth, determining a divergence by comparing the latent variables with a multi-dimensional Gaussian distribution, and adjusting parameters of the probabilistic encoders and the logistic regression decoder based on the cost and the divergence.

In at least some embodiments, training multi-modal models with batches of limited modality combinations further includes applying the model to a live data sample including data values corresponding to less than all of the plurality of data types. In at least some embodiments, the plurality of modality lists include even distributions of modalities among the plurality of modalities. In at least some embodiments, the generating the plurality of modality lists includes determining possible combinations of modalities. In at least some embodiments, listed modalities of each modality list among the plurality of modality lists are included in a predetermined number of combinations of modalities.

The foregoing outlines features of several embodiments so that those skilled in the art would better understand the aspects of the present disclosure. Those skilled in the art should appreciate that this disclosure is readily usable as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that various changes, substitutions, and alterations herein are possible without departing from the spirit and scope of the present disclosure.

What is claimed is:

1. A non-transitory computer-readable medium including instructions that, in response to execution by one or more processors, cause performance of operations comprising:
grouping training data samples among a plurality of training data samples into a plurality of batches of training data samples, each training data sample including data values corresponding to each of a plurality of data types;
grouping data types among the plurality of data types into a plurality of modalities of data types;
generating a plurality of modality lists, each modality list including one or more modalities; and
performing, for each modality list among the plurality of modality lists to produce a multi-modal model for estimating a result from an incomplete data sample:
applying, for each modality in the modality list, a probabilistic encoder to the data values corresponding to the modality in a batch of training samples among the plurality of batches to obtain a feature encoding corresponding to the modality, integrating the feature encoding of each modality in the modality list to produce latent variables,
applying a logistic regression decoder to the latent variables to estimate the result;
determining a cost by comparing the estimated result with a ground truth,
determining a divergence by comparing the latent variables with a multi-dimensional Gaussian distribution, and
adjusting parameters of the probabilistic encoders and the logistic regression decoder based on the cost and the divergence.

2. The computer-readable medium of claim 1, further comprising applying the multi-modal model to a live data sample including data values corresponding to less than all of the plurality of data types.

3. The computer-readable medium of claim 1, wherein the plurality of modality lists include even distributions of modalities among the plurality of modalities.

4. The computer-readable medium of claim 1, wherein the generating the plurality of modality lists includes determining possible combinations of modalities.

5. The computer-readable medium of claim 4, wherein listed modalities of each modality list among the plurality of modality lists are included in a predetermined number of combinations of modalities.

6. The computer-readable medium of claim 1, wherein the generating the plurality of modality lists includes storing the plurality of modality lists in a memory.

7. The computer-readable medium of claim 1, wherein the feature encoding represents an average and a variance.

8. The computer-readable medium of claim 1, wherein each training data sample among the plurality of training data samples corresponds to a person, and wherein each data type among the plurality of data types is a body measurement.

9. The computer-readable medium of claim 8, wherein the applying the logistic regression decoder to the latent variables is to estimate a risk of a lifestyle-related disease.

10. A method comprising:
grouping training data samples among a plurality of training data samples into a plurality of batches of training data samples, each training data sample including data values corresponding to each of a plurality of data types;
grouping data types among the plurality of data types into a plurality of modalities of data types;
generating a plurality of modality lists, each modality list including one or more modalities; and
performing, for each modality list among the plurality of modality lists to produce a multi-modal model for estimating a result from an incomplete data sample:
applying, for each modality in the modality list, a probabilistic encoder to the data values corresponding to the modality in a batch of training samples among the plurality of batches to obtain a feature encoding corresponding to the modality, integrating the feature encoding of each modality in the modality list to produce latent variables,
applying a logistic regression decoder to the latent variables to estimate the result;
determining a cost by comparing the estimated result with a ground truth,
determining a divergence by comparing the latent variables with a multi-dimensional Gaussian distribution, and
adjusting parameters of the probabilistic encoders and the logistic regression decoder based on the cost and the divergence.

11. The method of claim 10, further comprising applying the multi-modal model to a live data sample including data values corresponding to less than all of the plurality of data types.

12. The method of claim 10, wherein the plurality of modality lists include even distributions of modalities among the plurality of modalities.

13. The method of claim 10, wherein the generating the plurality of modality lists includes determining possible combinations of modalities.

14. The method of claim 13, wherein listed modalities of each modality list among the plurality of modality lists are included in a predetermined number of combinations of modalities.

15. The method of claim 10, wherein the generating the plurality of modality lists includes storing the plurality of modality lists in a memory.

16. A device comprising:
a controller including circuitry configured to perform operations comprising,
grouping training data samples among a plurality of training data samples into a plurality of batches of training data samples, each training data sample including data values corresponding to each of a plurality of data types;
grouping data types among the plurality of data types into a plurality of modalities of data types;
generating a plurality of modality lists, each modality list including one or more modalities; and
performing, for each modality list among the plurality of modality lists to produce a multi-modal model for estimating a result from an incomplete data sample:
applying, for each modality in the modality list, a probabilistic encoder to the data values corresponding to the modality in a batch of training samples among the plurality of batches to obtain a feature encoding corresponding to the modality,
integrating the feature encoding of each modality in the modality list to produce latent variables,
applying a logistic regression decoder to the latent variables to estimate the result;
determining a cost by comparing the estimated result with a ground truth,
determining a divergence by comparing the latent variables with a multi-dimensional Gaussian distribution, and
adjusting parameters of the probabilistic encoders and the logistic regression decoder based on the cost and the divergence.

17. The device of claim 16, further comprising applying the multi-modal model to a live data sample including data values corresponding to less than all of the plurality of data types.

18. The device of claim 16, wherein the plurality of modality lists include even distributions of modalities among the plurality of modalities.

19. The device of claim 16, wherein the generating the plurality of modality lists includes determining possible combinations of modalities.

20. The device of claim 19, wherein listed modalities of each modality list among the plurality of modality lists are included in a predetermined number of combinations of modalities.
